(19) United States
(12) Patent Application Publication
(10) Pub. No.: US 2015/0099706 A1
(43) Pub. Date: Apr. 9, 2015

(54) TREATMENT OF ISCHEMIC STROKE WITH DRa1-MOG-35-55

(71) Applicants: Oregon Health & Science University, Portland, OR (US); The United States Government as Represented by the Department of Veterans Affairs, Washington, DC (US)

(72) Inventors: Halina Offner-Vandenbark, Portland, OR (US); Nabil Alkayed, West Linn, OR (US); Arthur A. Vandenbark, Portland, OR (US)

(73) Assignees: Oregon Health & Science University; The United States Government as Represented by the Department of Veterans Affairs

(21) Appl. No.: 14/506,486

(22) Filed: Oct. 3, 2014

Related U.S. Application Data
(60) Provisional application No. 61/886,299, filed on Oct. 3, 2013.

(51) Int. Cl. A61K 38/17

(52) U.S. Cl. CPC A61K 38/1774; A61K 38/177
USPC 514/17.7

(57) ABSTRACT

Methods and compositions used in treating ischemic stroke using a recombinant DRα-MOG-35-55 construct are disclosed. The disclosed methods involve administering a pharmaceutical composition comprising DRα-MOG-35-55 and a pharmaceutically acceptable carrier to a subject that has had or is at risk of developing ischemic stroke.

### CROSS REFERENCE TO RELATED APPLICATIONS

This claims the benefit of U.S. Provisional Application No. 61/886,299, filed Oct. 3, 2013, which is incorporated herein by reference in its entirety.

### ACKNOWLEDGEMENT OF GOVERNMENT SUPPORT

This invention was made with United States government support under the terms of 1 RO1 NS076013 and 5 RO1 NS047661 awarded by the National Institutes of Health. The United States government has certain rights to this invention.

### FIELD

This disclosure relates methods of treating ischemic stroke with biological agents, particularly methods of treating ischemic stroke with partial MHC molecules.

### BACKGROUND

It is well established that experimental stroke triggers inflammation in the brain as well as rapid activation of the peripheral immune system, resulting in migration of monocytes, neutrophils and T-cells across the blood-brain barrier into the growing infarct and further activation of microglial cells (Gee et al., Stroke 38 2 Suppl, 783-788 (2007); Nilupul Perera et al., J Clin Neurosci 13, 1-8 (2006); Wang P Y et al., Stroke 24, 236-240 (1993); all of which are incorporated by reference herein). These infiltrating cells contribute to ischemic damage through localized inflammation. The magnitude of the inflammatory response is strongly associated with stroke outcome in patients (Emsley H C et al., Stroke 36, 228-229 (2005); Smith C J et al., BMC Neurol 4, 2 (2004); both of which are incorporated by reference herein). Furthermore, the peripheral immune system is massively activated after cerebral ischemia. This vast activation is followed by immunosuppression that is marked by atrophy of the spleen and thymus (Offner et al., J Cereb Blood Flow Metab 26, 654-665 (2006a); Offner et al., J Immunol 176, 6523-6531 (2006b); both of which are incorporated by reference herein). Immunotherapeutic approaches for treatment of ischemic stroke could therefore reduce the inflammatory milieu, target specific mechanisms of the inflammatory pathway and maintain homeostasis of peripheral immunity.

Recombinant T-cell receptor ligand (RTL) molecules consist of the α1 and β1 domains of MHC class II molecules expressed as a single polypeptide with or without antigenic amino terminal extensions (Burrows et al., Protein Eng 12, 771-778 (1999); Vandenbark et al., J Immunol 171, 127-133 (2003); both of which are incorporated by reference herein). It has been previously demonstrated that RTL could prevent and/or reverse clinical signs of experimental autoimmune encephalomyelitis (EAE) and that an RTL construct could effectively treat experimental stroke in mice (Akiyoshi K et al., Transl Stroke Res 2, 404-410 (2011); Burrows et al., J Immunol 161, 5987-5996 (1998); Burrows et al., J Immunol 167, 4386-4395 (2001); Huan et al., J Immunol 172, 4556-4566 (2004); Subramanian et al., Stroke 40, 2539-2545 (2009); Vandenbark et al., (2003) supra; all of which are incorporated by reference herein). Furthermore, a construct called RTL1000 that is comprised of an HLA-DR2 moiety linked to human MOG-35-55 peptide in humanized DR2 mice has been shown to reduce stroke infarct size (Akiyoshi et al. 2011).

Recently, it was shown that RTL1000 can directly bind to and downregulate the cell surface expression of the MHC class II invariant chain (CD74) on CD11b+ monocytes, inhibit binding of macrophage migration inhibitory factor (MIF) to CD74 and block downstream inflammatory effects in the CNS (Benedek G et al., Eur J Immunol 43, 1309-1321 (2013); Vandenbark et al., J Autoimmun 40, 96-110, (2013); both of which are incorporated by reference herein.

### SUMMARY

Treatment with DRα1-MOG-35-55 after the onset of middle cerebral artery occlusion (MCAO) reduces infarct size, inhibits infiltration of activated monocytes into the ischemic brain and reverses splenic atrophy, which is typically induced after MCAO.

Disclosed herein are methods of treating ischemic stroke in a subject that involve administering to the subject an effective amount of a pharmaceutical composition comprising DRα1-MOG-35-55, alone or in combination with another active composition in a pharmaceutically acceptable carrier. In some examples, the method can involve administering the composition after the onset of ischemia. In other examples, the composition can comprise a dose of DRα1-MOG-35-55 between 4 mg/kg and 25 mg/kg given on 4 consecutive days. In still other examples, the composition may be formulated for subcutaneous or intravenous administration

### BRIEF DESCRIPTION OF THE DRAWINGS

FIG. 1A is a bar graph indicating infarct volumes in mice treated with 500 μg/ml DRα1-MOG-35-55 (black bars, n=10) versus vehicle-treated (white bars, n=10) mice after 60 min MCAO and 96 hr of reperfusion in cortex (CTX), caudate-putamen (CP), and total hemisphere (HMSPHR) (*p<0.05).

FIG. 1B is a set of images of representative 2,3,5-triphenyltetrazolium chloride stained cerebral sections after 96 hr of reperfusion following 1 hr of MCAO.

FIG. 2A is a bar graph representing the total number of lymphocytes per brain recovered from the non-ischemic left and ischemic right brain from mice treated with 500 μg/ml DRα1-MOG-35-55 (n=7; black bars) versus vehicle-treated mice (n=9; white bars).

FIG. 2B is a bar graph of the total number of CD11b⁺CD45^{low}, CD11b⁺CD45^{high} and CD4⁺ cells per brain recovered from the ischemic right brains of DRα1-MOG-35-55-treated (n=7; black bars) versus Vehicle-treated mice (n=9; white bars).

FIG. 2C is a bar graph of the expression of CD74 on CD11b⁺CD45^{high} cells recovered from ischemic right brains of DRα1-MOG-35-55-treated (n=7; black bars) versus vehicle-treated mice (n=9; white bars).

For all of FIGS. 2A, 2B and 2C, data are presented as mean±SEM. *p<0.05, **p<0.01, ***p<0.001 by Student's t-test.

FIG. 3 is a set of four bar graphs indicating the relative expression of mRNA of inflammatory genes was analyzed by real-time PCR from pooled ischemic brain samples of DRα1-MOG-35-55-treated (n=3) relative to vehicle-treated (n=3) mice. Values <1 indicate genes down-regulated in DRα1-MOG-35-55-treated mice relative to vehicle treated mice. Values=1 indicate the same expression in DRα1-MOG-35-55-treated mice relative to vehicle treated mice. Values >1 indicate genes that are up-regulated in DRα1-MOG-35-55-treated mice relative to vehicle treated mice. ND: Expression was not detected.

FIG. 4A is a bar graph of total cell number per spleen from DRα1-MOG-35-55-treated (black bars, n=10) versus vehicle-treated mice (white bars, n=12).

FIG. 4B is a bar graph of the number of CD11b⁺ and CD4⁺ cells per spleen from DRα1-MOG-35-55-treated (black bars, n=10) versus vehicle-treated mice (white bars, n=12).

FIG. 4C is a bar graph of the percent of CD11b+ and CD4+ cells in spleens from DRα1-MOG-35-55-treated (black bars, n=10) versus vehicle-treated mice (white bars, n=12).

For all of FIGS. 4A, 4B, and 4C, data are presented as mean±SEM. *p<0.05, **p<0.01 Student's t-test.

FIG. 5A is a bar graph of the expression of CD80, HLA MHC class II, ICAM-1 and CCR2 on CD11b⁺ cells recovered from the spleens of DRα1-MOG-35-55-treated (n=10; black bars) and vehicle-treated mice (n=12; white bars).

FIG. 5B is a bar graph of the expression of CD44 and CD62L on CD4⁺ cells recovered from the spleens of DRα1-MOG-35-55-treated (n=10; black bars) and vehicle-treated mice (n=12; white bars).

For FIGS. 5A and 5B, data are presented as mean±SEM. *p<0.05, by Student's t-test.

FIG. 6 is a set of four bar graphs of mRNA expression of inflammatory genes by real time PCR from pooled spleen samples collected from DRα1-MOG-35-55-treated (n=6) and vehicle-treated (n=6) mice. Values <1 indicate genes down-regulated in DRα1-MOG-35-55-treated mice relative to vehicle treated mice. Values=1 indicate the same expression in DRα1-MOG-35-55-treated mice relative to vehicle treated mice. Values >1 indicate genes that are upregulated in DRα1-MOG-35-55-treated mice relative to vehicle treated mice. ND: Expression was not detected.

FIG. 7 is a bar graph showing the number of CD11b⁺ CD45^{low} and CD11b⁺ CD45^{high} cells recovered from the non-ischemic left brains of DRα1-MOG-35-55-treated (n=7, spotted bars) and vehicle treated (n=9, white bars) mice. Data are presented as mean±SEM.

FIG. 8 is a set of three bar graphs showing the relative mRNA expression of the three indicated genes by real time PCR from ischemic brain samples of DRα1-MOG-35-55-treated (n=3, spotted bars) and vehicle treated (n=3, white bars) mice. *−p<0.05 by Student's t-test.

FIG. 9 is a bar graph showing TNFα protein production in CD11b+ and CD4+ cell subsets by intracellular staining in DRα1-MOG-35-55-treated (spotted bars) and vehicle treated (white bars) treated mice. Splenocytes were collected, cultured, and stimulated with 50 ng/ml PMA, 500 ng/ml ionomycin, and 10 μg/ml Brefeldin A for four hours.

FIG. 10 is a set of three bar graphs showing the relative mRNA expression of the three indicated genes by real time PCR from ischemic brain samples of DRα1-MOG-35-55-treated (n=6, spotted bars) and vehicle treated (n=6, white bars) mice. *−p<0.01 by Student's t-test.

### SEQUENCE LISTING

Any nucleic acid and amino acid sequences listed herein or in the accompanying sequence listing are shown using standard letter abbreviations for nucleotide bases and amino acids, as defined in 37 C.F.R. §1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Oct. 2, 2014, and is 1590 bytes, which is incorporated by reference herein.

SEQ ID NO: 1 is the amino acid sequence of an exemplary DRα1-MOG-35-55 with the MOG peptide conjugated to the N-terminus.

### DETAILED DESCRIPTION

Disclosed herein are methods for treating stroke (for example, ischemic stroke) in a subject. In some embodiments, the disclosed methods include administering to a subject (such as a subject who has had, is having, or is at risk for stroke) an effective amount of a disclosed DRα1-MOG-35-55 polypeptide, for example, a composition including the polypeptide. In particular examples, the DRα1-MOG-35-55 polypeptide is chimeric protein construct comprising: a) a human DRα1 polypeptide, b) a MOG-35-55 peptide (such as human MOG-35-55 or mouse MOG-35-55), and c) peptide linker. The peptide linker comprises a first glycine-serine spacer, a thrombin cleavage site and a second glycine-serine spacer. The linker is covalently bound to the amino terminus of the DRα1 polypeptide and to the carboxyl terminus of a MOG-35-55 peptide. In one example, the DRα1-MOG-35-55 polypeptide includes or consists of the amino acid sequence of SEQ ID NO: 1.

One of skill in the art can determine effective amounts of a DRα1-MOG-35-55 polypeptide for administration to a subject to treat stroke, for example, based on studies in vitro or in animal models (such as the mouse model of stroke described in the Examples). In some embodiments, the polypeptide is administered to a subject (such as a human subject) in an amount from about 0.1 mg/kg to about 100 mg/kg (such as about 0.5 mg/kg to about 10 mg/kg, about 4 mg/kg to about 25 mg/kg, about 2 mg/kg to about 50 mg/kg, or about 10 mg/kg to about 100 mg/kg). In other examples, a subject is administered a unit dose of the DRα1-MOG-35-55 polypeptide, such as about 0.1 mg, 0.5 mg, 1 mg, 2 mg, 5 mg, 10 mg, 20, mg, 30 mg, 40 mg, 50 mg, 60 mg, 75 mg, 100 mg, 200 mg, 300 mg, 400 mg, 500 mg, 1 g, 2 g, or more.

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.”

In addition, the materials, methods, and examples are illustrative only and not intended to be limiting. In order to facilitate review of the various embodiments of the disclosure, the following explanations of specific terms are provided:

Administration: To provide or give a subject an agent, such as a pharmaceutical composition comprising DRα1-MOG-35-55 by any effective route. Exemplary routes of administration include, but are not limited to parenteral injection, such as intravenous, subcutaneous, or intraperitoneal injection.

Effective amount: An amount of agent, such as DRα1-MOG-35-55, that is sufficient to generate a desired response, such as the reduction or elimination of a sign or symptom of a condition or disease, such as ischemia/reperfusion injury caused by ischemic stroke. Alternatively, an effective amount may be an amount sufficient to generate a desired response in a cell or cell type, such as an effective amount to protect a neuron or other cell of the nervous system from damage resulting from ischemia/reperfusion injury.

When administered to a subject, a dosage will generally be used that will achieve target tissue concentrations that have been shown to achieve activity in vitro. In some examples, an “effective amount” is one that prophylactically treats one or more symptoms and/or underlying causes of a disorder or disease. An effective amount can also be an amount that therapeutically treats one or more symptoms and/or underlying causes of a disorder or disease.

Treating a disease: Inhibiting the full development of a disease or condition, for example, in a subject who is at risk for a disease such as ischemic stroke and/or ischemia/reperfusion injury caused by ischemic stroke. Treatment refers to any therapeutic intervention that ameliorates a sign or symptom of a disease or pathological condition. The term “ameliorating,” with reference to a disease or pathological condition, refers to any observable beneficial effect provided by a pharmaceutical composition. The beneficial effect can be evidenced, for example, by a delayed onset of clinical symptoms of the disease in a susceptible subject, a reduction in severity of some or all clinical symptoms of the disease, a slower progression of the disease or by other clinical or physiological parameters associated with a particular disease. A “prophylactic” treatment is a treatment administered to a subject who does not exhibit signs of a disease or exhibits only early signs for the purpose of decreasing the risk of developing pathology. For example, a prophylactic dose of DRα1-MOG-35-55 can be administered to a subject undergoing heart surgery for the prevention of the ischemic/reperfusion injury that is a common side effect of such surgeries. A therapeutic treatment is a treatment administered to a subject who has already exhibited signs or symptoms of a disease.

Subject: A living multicellular vertebrate organism, a category that includes, for example, mammals and birds. A “mammal” includes both human and non-human primates (such as monkeys), as well as non-primate mammals such as mice or rabbits any other research animals. In some examples, a subject is a human patient, such as a patient that has had or is at risk of developing an ischemic event.

Ischemia/stroke: Ischemia may be any reduction in the flow of oxygenated blood to a tissue or organ. Similarly, an ischemic event may be any event, action, process, injury, or other disruption that results in decreased blood flow to a cell, collection or group of cells, tissue, or organ. Examples of ischemic events include vasoconstriction, thrombosis and embolism.

A stroke may be any interruption of blood flow to any part of the brain. A stroke can be due to an ischemic event (for example, occlusion of a blood vessel due to a thrombus or an embolism) or hemorrhage (for example of a cerebral blood vessel). Any of these events may result in hypoxia. In stroke, ischemia has detrimental effects on neural cells. A neural cell may be any cell derived from a lineage that originates with a neural stem cell and includes a mature neuron. Thus, the term neural cell includes neurons (nerve cells) as well as their progenitors regardless of their stage of differentiation. In the context of an adult brain, neural cells are predominantly differentiated neurons. In one example, neural cells include hippocampal neurons and cortical neurons. In contrast, a non-neural cell may be any cell derived from any lineage other than a neural cell lineage. For example, it may be any cell does not terminally differentiate into a mature neuron. Some non-neural cells make up part of the central nervous system (CNS). Examples of non-neural cells in the CNS include cells of the brain (such as glial cells and immune system cells, such as B cells, dendritic cells, macrophages and microglia).

A subject may be considered at risk of stroke and/or if there is an increased probability that the subject will undergo an event resulting in ischemia/reperfusion injury relative to the general population. Accordingly, risk is a statistical concept based on empirical and/or actuarial data. Commonly, risk can be correlated with one or more indicators, such as symptoms, signs, characteristics, properties, occurrences, events or undertakings, of a subject. For example, with respect to hypoxic injury in the brain resulting from ischemia, indicators include but are not limited to high blood pressure (hypertension), atrial fibrillation, transient ischemic events, prior stroke, diabetes, high cholesterol, angina pectoris, and heart disease.

Additional risk indicators for hypoxic injury include surgery, especially cardiovascular surgeries, such as endarterectomy, pulmonary bypass surgery or coronary artery bypass surgery. Additional risk factors or indicators include non-medical activities, such as motorcycle riding, contact sports and combat operations. Other risk factors are discussed herein, and yet more can be recognized by those of ordinary skill in the art.

Innate and adaptive immunity play an important role for the outcome after focal cerebral ischemia (stroke). Focal cerebral ischemia elicits a strong inflammatory response involving early recruitment of granulocytes and delayed infiltration of ischemic areas and the boundary zones by T cells and macrophages. (Stoll G et al, Neurobiology 56, 149-171 (1998).

Within hours of a stroke, transcription factors such as nuclear factor KB are activated locally in the brain tissue. These transcription factors upregulate proinflammatory genes including TNFα, interleukin 1β, interleukin 6, and IL-1 receptor agonist and chemokines such as IL-8, interferon inducible protein-10 and monocyte chemoattractant protein-1 (O'Neill L A et al, Trends Neurosci 20, 252-258 (1997), Liu et al. Stroke 25, 1481-1418 (1994), Wang et al, Mol Chem Neuropathol 23, 103-114 (2004), Wang et al, Stroke 26, 661-666 (1995), Wang et al, J Cereb Blood Flow Metab 15, 166-171 (1995), Wang et al., Stroke 28, 155-162 (1997), Kim et al, Neuroimmunol 56, 127-34 (1995), Wang et al, J Neurochem 71, 1194-1204 (1998)). These factors promote expression of adhesion molecules by vascular endothelial cells that allow infiltration into the brain of blood neutrophils, monocytes, macrophages and T cells that promote further brain injury. (Barone, F C et al, Cerebral Blood Flow Metab 19, 819-834 (1999). Additionally, inflammatory and antigenic products derived from the brain such as myelin basic protein may leak across a damaged blood brain barrier and produce reciprocal system activation (Offner et al, J of Cerebral Blood Flow & Metabolism 26, 654-655 (2006).

II. Pharmaceutical Compositions comprising DRα1-MOG-35-55

DRα1-MOG-35-55 can be combined with a pharmaceutically acceptable carrier appropriate for the particular route of administration being employed. One of skill in the art in light of this disclosure would understand how to combine DRα1-MOG-35-55 with the appropriate carrier for use in a particular route of administration. Dosage forms of DRα1-MOG-35-55 include excipients recognized in the art of pharmaceutical compounding as being suitable for the preparation of dosage units as discussed below. Such excipients include, without intended limitation, binders, fillers, lubricants, emulsifiers, suspending agents, sweeteners, flavorings, preservatives, buffers, wetting agents, disintegrants, effervescent agents and other conventional excipients and additives.

Compositions comprising DRα1-MOG-35-55 can thus include any one or combination of the following: a pharmaceutically acceptable carrier or excipient; other medicinal agent(s); pharmaceutical agent(s); adjuvants; buffers; preservatives; diluents; and various other pharmaceutical additives and agents known to those skilled in the art. These additional formulation additives and agents can be biologically inactive and can be administered to patients without causing deleterious side effects or interactions with DRα1-MOG-35-55.

DRα1-MOG-35-55 can be administered in a controlled release form by use of a slow release carrier, such as a hydrophilic, slow release polymer. Exemplary controlled release agents in this context include, but are not limited to, hydroxypropyl methyl cellulose, having a viscosity in the range of about 100 cps to about 100,000 cps or other biocompatible matrices such as cholesterol.

Pharmaceutical compositions comprising DRα1-MOG-35-55 may be formulated for use in parenteral administration, e.g. intravenously, intramuscularly, subcutaneously or intraperitoneally, including aqueous and non-aqueous sterile injection solutions which may optionally contain anti-oxidants, buffers, bacteriostats and/or solutes which render the formulation isotonic with the blood of the mammalian subject; and aqueous and non-aqueous sterile suspensions which may include suspending agents and/or thickening agents. The formulations may be presented in unit-dose or multi-dose containers.

The parenteral preparations may be solutions, dispersions or emulsions suitable for such administration. Pharmaceutically parenteral formulations and ingredients thereof are sterile or readily sterilizable, biologically inert, and easily administered. Pharmaceutically acceptable carriers used in parenteral formulations comprising DRα1-MOG-35-55 of are well known to those of ordinary skill in the pharmaceutical compounding arts. Parenteral preparations typically contain buffering agents and preservatives, and injectable fluids that are pharmaceutically and physiologically acceptable such as water, physiological saline, balanced salt solutions, aqueous dextrose, glycerol or the like. Injection solutions, emulsions and suspensions may be prepared from sterile powders, granules and tablets of the kind previously described. Unit dosage formulations are those containing a daily or other dose or unit, daily sub-dose, as described herein above, or an appropriate fraction thereof, of the active ingredient(s), including a dose between 20 and 25 mg/kg.

III. Administration of Pharmaceutical Compositions comprising DRα1-MOG-35-55

Suitable routes of administration of DRα1-MOG-35-55 polypeptide include, but are not limited to, oral, buccal, nasal, aerosol, topical, transdermal, mucosal, injectable, slow release, controlled release, iontophoresis, sonophoresis, and other conventional delivery routes, devices and methods. Injectable delivery methods include, but are not limited to, intravenous, intramuscular, intraperitoneal, intraspinal, intrathecal, intracerebroventricular, intraarterial, intranasal and subcutaneous injection.

Amounts and regimens for the administration of DRα1-MOG-35-55 to a subject can be determined by one of skill in the art. Typically, the dose range will be from about 0.1 μg/kg body weight to about 100 mg/kg body weight. Other suitable ranges include doses of from about 100 μg/kg to 1 mg/kg body weight. In certain embodiments, the effective dosage will be selected within narrower ranges of, for example, 5-40 mg/kg, 10-35 mg/kg or 20-25 mg/kg. These and other effective unit dosage amounts may be administered in a single dose, or in the form of multiple daily, weekly or monthly doses, for example in a dosing regimen comprising from 1 to 5, or 2-3, doses administered per day, per week, or per month. The dosing schedule may vary depending on a number of clinical factors, such as the subject's sensitivity to the protein. One example of a dosing schedule for ischemic stroke is 20-25 mg/kg administered within 4 hours of an ischemic event, followed by daily dosing for 1, 2, 3, or 4 or more days following the ischemic event.

IV. Combinatorial Formulations and Co-Administration of DRα1-MOG-35-55 with other Compositions

DRα1-MOG-35-55 may also be formulated with other pharmaceutical compositions such that co-administration of DRα1-MOG-35-55 and one or more additional active agent may be employed in the treatment of ischemic stroke. Exemplary combinatorial formulations and coordinate treatment methods in this context employ a purified partial MHC polypeptide in combination with one or more additional or adjunctive therapeutic agents. The secondary or adjunctive methods and compositions useful in the treatment of inflammatory diseases include, but are not limited to, immunoglobulins, copolymer 1, copolymer 1-related peptides, and T-cells treated with copolymer 1 or copolymer 1-related peptides (see, e.g., U.S. Pat. No. 6,844,314, incorporated herein by reference); blocking monoclonal antibodies, transforming growth factor-α, entanercept or anti-TNF α antibodies; anti-coagulants including but not limited to, warfarin, heparin; anti-platelet medications including but not limited to aspirin, clopidogrel or aggrenox; clot dissolving medications including, but not limited to tissue plasminogen activating factor (tPA); angiotensin-converting enzyme (ACE) inhibitors, including but not limited to benazepril, captopril, enalapril, fosinopril, lisinopril, perindopril, quinapril, ramipril, and trandolapril; angiotensin II receptor blockers (ARBs) including but not limited to candesartan cilexetil, eprosartan mesylate, irbesartan, losartan, olmesartan, telmisartan, or valsartan; beta-blockers including but not limited to acebutolol, atenolol, betaxolol, carvedilol, labetalol, metoprolol, nadolol, penbutolol, pindolol, propranolol, timolol; diuretics including but not limited to chlorthalidone and chlorthalidone combinations, chlorothiazide, hydrochlorothiazide and hydrochlorothiazide combinations, indapamide, bumetanide, furosemide, torsemide, amiloride, spironolactone and spironolactone combinations, triamterene and triamterene combinations, metolazone; and calcium channel blockers including but not limited to amlodipine, amlodipine and atorvastatin, amlodipine and benazepril hydrochloride, diltiazem, enalapril maleate-felodipine ER, felodipine, isradipine, nicardipine, nifedipine, nisoldipine, verapamil; neuroprotectants; statins; anti-inflammatory agents; immunosuppressive agents; alkylating agents; anti-metabolites; antibiotics; corticosteroids; proteosome inhibitors; diketopiperazines; and steroidal agents including but not limited to estrogens, progesterones, testosterones, corticosteroids, and anabolic steroids.

To practice the coordinate administration methods of the invention, DRα1-MOG-35-55 is administered simultaneously or sequentially with one or more secondary or adjunctive therapeutic agents. The coordinate administration may be done in either order, and there may be a time period while only one or both (or all) active therapeutic agents, individually and/or collectively, exert their biological activities. The coordinate administration of DRα1-MOG-35-55 with a secondary therapeutic agent as contemplated herein can yield an enhanced therapeutic response beyond the therapeutic response elicited by either or both the purified MHC polypeptide and/or secondary therapeutic agent alone. The enhanced therapeutic response may allow for lower doses of DRα1-MOG-35-55 and/or the secondary therapeutic agent.

The use of DRα1-MOG-35-55 alone or in combination with other therapeutic agents may also be accompanied by physical intervention such as, for example, angioplasty, stents, carotid endarterectomy, revascularization and endovascular surgery.

### EXAMPLES

The following examples are illustrative of disclosed methods. In light of this disclosure, those of skill in the art will recognize that variations of these examples and other examples of the disclosed method would be possible without undue experimentation.

### Example 1

### Materials and Methods

Animals: All experiments used age-matched, sexually mature (20 to 25 g) male HLA-DRB1*1502 (DR2-Tg) mice described in Gonzalez-Gay et al, Hum Immunol 50, 54-60 (1996); incorporated by reference herein.

HLA-DRα1-MOG-35-55 cloning, production and purification: Briefly, DRα1-MOG-35-55 was constructed using a DRα1 construct as a template. The mouse MOG (35-55) peptide DNA encoding sequence was attached to the N-terminus of the DRα1 domain with a linker-thrombin-linker intervening element.

Treatment with DRα1-MOG-35-55: Mice were randomized to receive 500 μg DRα1-MOG-35-55 in a 0.1 ml volume or 0.1 ml Vehicle (5% dextrose in Tris-HCl, pH 8.5) by subcutaneous injection 4 hours after the onset of reperfusion followed by similar doses at 24, 48, and 72 hr of reperfusion for a total of 4 treatments each of DRα1-MOG-35-55 or Vehicle. Both DRα1-MOG-35-55 and Vehicle treated MCAO mice were euthanized at the 96 hour time-point for evaluation of tissues and cells.

Transient Middle Cerebral Artery Occlusion: Transient focal cerebral ischemia was induced in male DR2-Tg mice for 1 hour by reversible right MCAO under isoflurane anesthesia followed by 96 hours of reperfusion, as previously described in Offner et al. (2006a, supra), with slight modifications. Head and body temperature were controlled at 36.5±0.5° C. throughout MCAO surgery with warm water pads and a heating lamp. Laser-Doppler flowmetry (LDF; Model DRT4, Moor Instruments Ltd., Oxford, England) was monitored throughout the ischemic period with a LDF probe affixed to the skull to ensure effective occlusion and reperfusion. The common carotid artery was exposed and the external carotid artery was ligated and cauterized. Unilateral MCAO was accomplished by inserting a 6-0 nylon monofilament surgical suture (ETHICON, Inc., Somerville, N.J., USA) with a heat-rounded and silicone-coated (Xantopren comfort light, Heraeus, Germany) tip into the internal carotid artery via the external carotid artery stump. Animals were excluded if mean intra-ischemic LDF was greater than 30% pre-ischemic baseline. At 1 hour of occlusion, the occluding filament was withdrawn to allow for reperfusion. Mice were then allowed to recover from anesthesia and survived for 96 hours following initiation of reperfusion.

Determination of Infarct Size: Brains were harvested after 96 hours of reperfusion and sliced into four 2-mm-thick coronal sections for staining with 1.2% 2,3,5-triphenyltetrazolium chloride (TTC; Sigma, St. Louis, Mo., USA) in saline as described in Hurn et al., J Cereb Blood Flow Metab 27, 1798-1805 (2007) which is incorporated by reference herein. The 2-mm brain sections were incubated in 1.2% TTC for 15 minutes at 37° C., and then fixed in 10% formalin for 24 hours. Infarct volume was measured using digital image analysis software (Systat, Inc., Point Richmond, Calif., USA). To control for edema, infarct volume (cortex, striatum, and hemisphere) was calculated by subtraction of the ipsilateral non-infarcted regional volume from the contralateral regional volume. This value was then divided by the contralateral regional volume and multiplied by 100 to yield regional infarct volume as a percent of the contralateral region.

Leukocyte isolation from brain and spleen: Spleens from MCAO-treated mice were removed and a single-cell suspension was prepared by passing the tissue through a 100 μm nylon mesh (BD Falcon, Bedford, Mass.). The cells were washed using RPMI 1640 and the red cells lysed using 1× red cell lysis buffer (eBioscience, Inc., San Diego, Calif.) and incubated for 3 minutes. The cells were then washed twice with RPMI 1640, counted, and resuspended in stimulation medium (RPMI, containing 2% FBS, 1% sodium pyruvate, 1% L-glutamine, 0.4% βME). The brain was divided into the ischemic (right) and nonischemic (left) hemispheres, digested for 60 minutes with 1 mg/ml Type IV collagenase (Sigma Aldrich, St. Louis, Mo.) and DNase I (50 mg/ml, Roche Diagnostics, Indianapolis, Ind.) at 37° C. with shaking at 200 rpm. Samples were mixed every 15 min. The suspension was washed 1× in RPMI, resuspended in 80% Percoll overlaid with 40% Percoll and centrifuged for 30 min at 1600 RPM. The cells were then washed twice with RPMI 1640, counted, and resuspended in staining medium.

Flow cytometry: Four-color (FITC, PE, APC and PerCP) fluorescence flow cytometry analyses were performed to determine the phenotypes of cells following standard antibody staining procedures. One million cells were washed with staining medium (PBS containing 0.1% NaN3 and 1% bovine serum albumin (Sigma, Illinois)) and incubated with combinations of the following monoclonal antibodies: CD80 (16-10A1), HLA-DR (TU39), CD11b (MAC-1), CD74 (In-1), CD45 (Ly-5), CD62L (MEL14), ICAM-1 (3B2), and CD44 (IM7), CCR2 (475301), CD4 (GK1.5) for 20 min at 4° C. Propidium iodide was added to identify dead cells. Data were collected with CELLQUEST (BD Biosciences, San Jose, Calif.) and FCS EXPRESS (De Novo Software, Los Angeles, Calif.) software on a FACSCalibur (BD Biosciences).

Intracellular staining for TNF-α: Splenocytes were resuspended (2×10⁶ cells/nil) in stimulation media (RPMI 1640 media containing 2% FCS, 1 mM pyruvate, 200 μg/ml penicillin, 200 U/ml streptomycin, 4 mM L-Glutamine, and 5×10⁻⁵ M 2-β-ME with PMA (50 ng/ml), ionomycin (500 ng/ml), and Brefeldin A (10 μg/ml); all reagents from BD Bioscience) for 4 hours. Fc receptors were blocked with mouse Fc receptor-specific mAb (2.3G2; BD PharMingen) before cell-surface staining and then fixed and permeabilized using a Cytofix/Cytoperm kit (BD Biosciences) according to the manufacturer's instructions. Permeabilized cells were washed with 1× Permeabilization Buffer (BD Bioscience) and stained with either PE-conjugated TNF-α (MP6-XT22) or isotype matched mAb that served as a negative control. Data were collected with CELLQUEST (BD Biosciences, San Jose, Calif.) and FCS EXPRESS (De Novo Software, Los Angeles, Calif.) software on a FACSCalibur (BD Biosciences).

Real time PCR: Splenocytes or brain cells were isolated from DR*1502-Tg mice. Total RNA was isolated from cells using an RNeasy® cultured cell kit according to the manufacturer's instructions. (Qiagen, Valencia, Calif., USA). Quantitative real time PCR was performed using the ABI7000 sequence detection system with gene-on-demand assay products (Applied Biosystems) for TaqMan array mouse immune response or for IL-4 (Assay ID: Mm00445259_m1), ACE (Assay ID: Mm00802048_m1), and CCL3 (Assay ID: Mm00441249_g1). GAPDH housekeeping gene was amplified as an endogenous control. Primers were used according to manufacturer's instructions.

Statistical Analysis: Data are presented as mean+SEM. Statistical differences in cortical, striatal, and total (hemispheric) infarct volume, as well as spleen and brain cell counts and percentages of cellular subtypes from FACS analyses were determined by Student's t-test. Statistical significance is defined as p<0.05.

### Example 2

### DRα1-MOG-35-55 Treatment Significantly Reduces Infarct Size after MCAO in DR2-Tg Mice

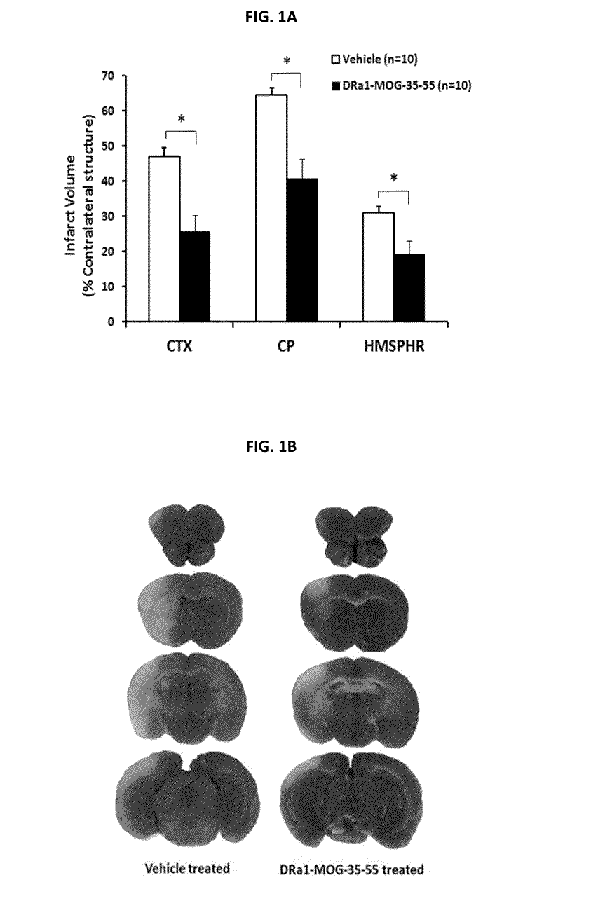

Evaluation of brain infarcts 96 hours after MCAO demonstrated that DRα1-MOG-35-55-treated male DR2-Tg mice had significantly reduced infarct volumes compared with the vehicle-treated group. Results are shown in FIG. 1A. Cortical infarct volume was 25.9±4.7% in DRα1-MOG-35-55-treated mice compared to 47.0±2.5% in vehicle-treated mice (p<0.01). Striatal infarct volume was 40.8±5.4% in DRα1-MOG-35-55-treated vs. 64.5±2.0% in vehicle-treated mice (p<0.01). The total hemispheric infarct volume was 19.4±3.6% in DRα1-MOG-35-55-treated vs. 31.1±1.7% in vehicle treated mice (p<0.01).

A quantitative assay of TTC stained cerebral sections after 96 hours of reperfusion confirmed the smaller infarct area in DRα1-MOG-35-55-treated mice compared with vehicle-treated mice (FIG. 1B). There were no significant differences in laser-Doppler perfusion before, during or immediately after MCAO between DRα1-MOG-35-55-treated and vehicle-treated groups.

### Example 3

### DRα1-MOG-35-55 Reduces the Number of Activated Microglia and Infiltrating Monocytes and their CD74 Cell Surface Expression in the Ischemic Brain

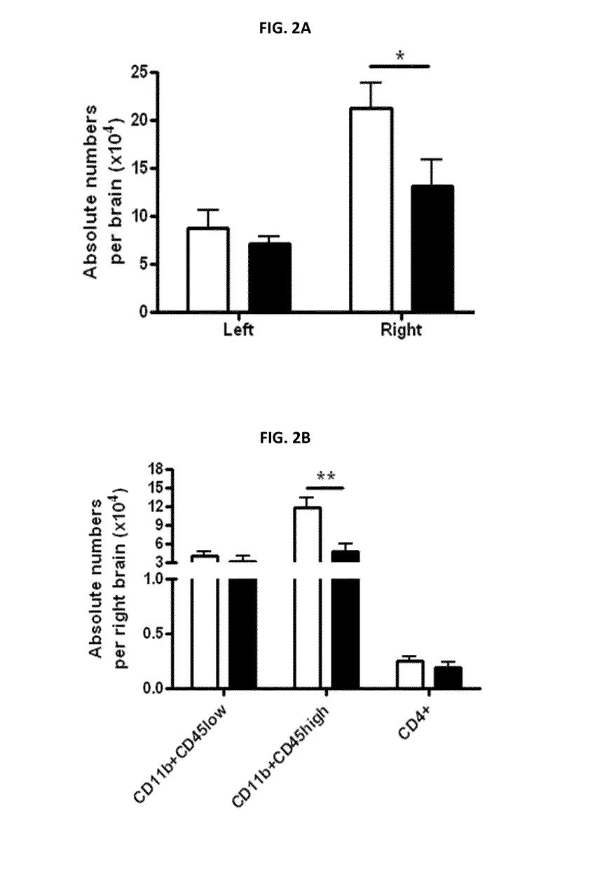

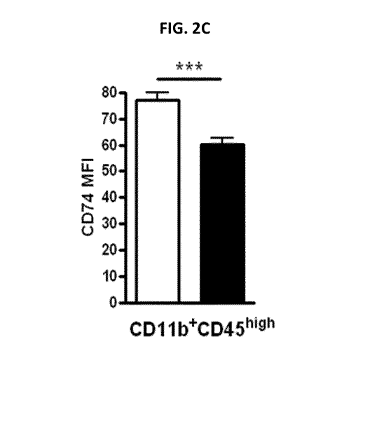

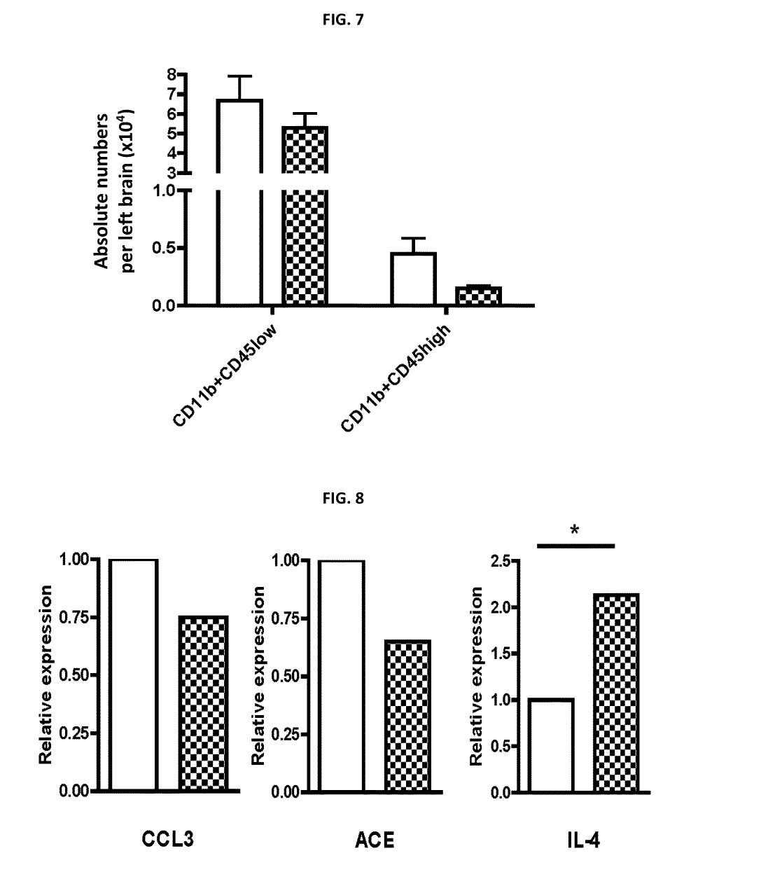

DRα1-MOG-35-55 treatment significantly reduced the absolute number of mononuclear cells in the right ischemic brain compared with vehicle-treated mice (13.14×10⁴ vs. 21.33×10⁴ respectively, p<0.05) (FIG. 2A). This difference is attributed mainly to the reduction in the number of activated CD11b⁺CD45^{high} monocytic cells (4.7×10⁴ vs. 11.85×10⁴ respectively, p<0.01). There were no significant differences in the absolute numbers of the CD4⁺ T cells or the resting microglia (CD11b⁺CD45^{low}) (FIG. 2B). In the non-ischemic left brain there were no differences in the total absolute number of mononuclear cells or in any cell type (FIG. 7).

Treatment of EAE with DRα1 lacking a conjugated MOG-35-55 peptide also leads to reduction of activated CD11b⁺ cells in the Central Nervous System (CNS) and DRα1 reduces the cell surface expression of the MIF receptor, CD74, on the activated CD11b⁺ cells. In order to determine if DRα1-MOG-35-55 treatment has the same effect in MCAO, expression of CD74 cell surface levels was evaluated on CD11b⁺CD45^{high} cells. As shown in FIG. 2C, there was a significant reduction in the level of CD74 expression as measured by the Mean Fluorescent Intensity (MFI) in the DRα1-MOG-35-55-treated mice compared with the Vehicle-treated mice (p<0.01).

### Example 4

### DRα1-MOG-35-55 Treatment Affects the Immune Gene Expression Profile in the Ischemic Brain after MCAO

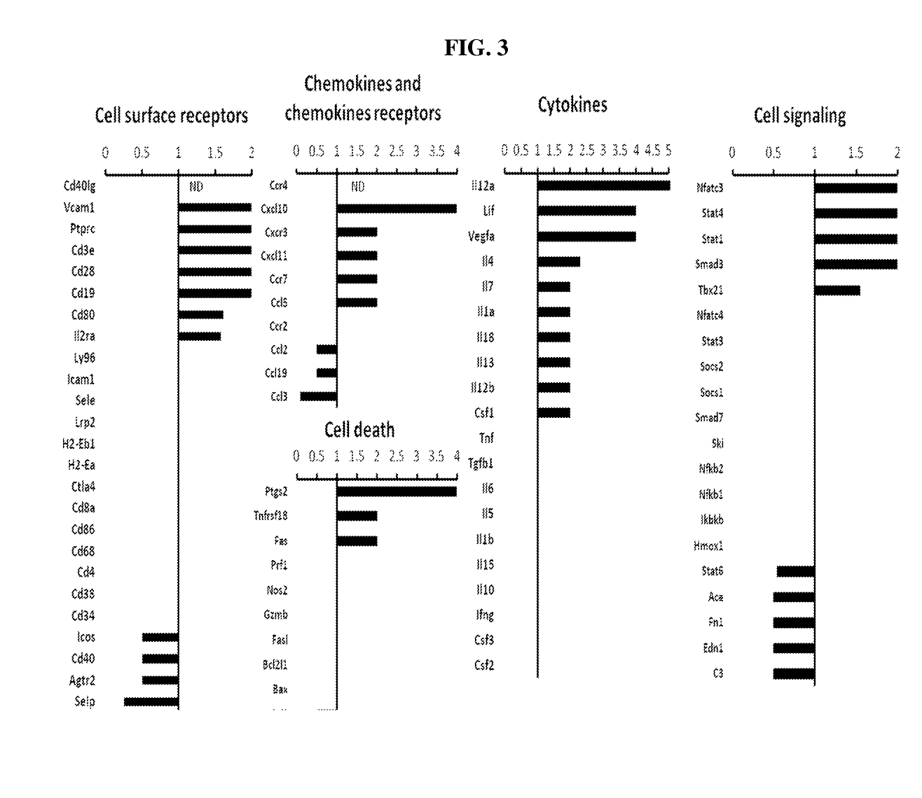

In order to assess the effect of DRα1-MOG-35-55 treatment on the expression of immune related genes in brain, mRNA was isolated from the ischemic brains of 3 vehicle-treated mice and 3 DRα1-MOG-35-55-treated mice. A real-time PCR assay was performed on pooled cDNA samples and expression levels of the DRα1-MOG-35-55-treated sample was analyzed relative to the Vehicle-treated sample (FIG. 3). Validation of 3 genes: IL-4, CCL3 and ACE, using individual samples, demonstrated that the expression trends were the same as in the immune array, although not all of the genes showed a significant difference (FIG. 8). The immune array data demonstrate that there was a decrease in the expression of monocyte-related genes such as CCL3, CCL2 and increases in Th1 and Th2 related genes such as IL-12, Tbx21, IL-4 and IL-13. It is important to note that several genes that were associated previously with cerebrovascular function and ischemic brain injury, including ACE and EDN1, were down regulated after DRα1-MOG-35-55 treatment relative to Vehicle treatment.

### Example 5

### DRα1-MOG-35-55 Treatment Reverses MCAO-Induced Splenic Atrophy

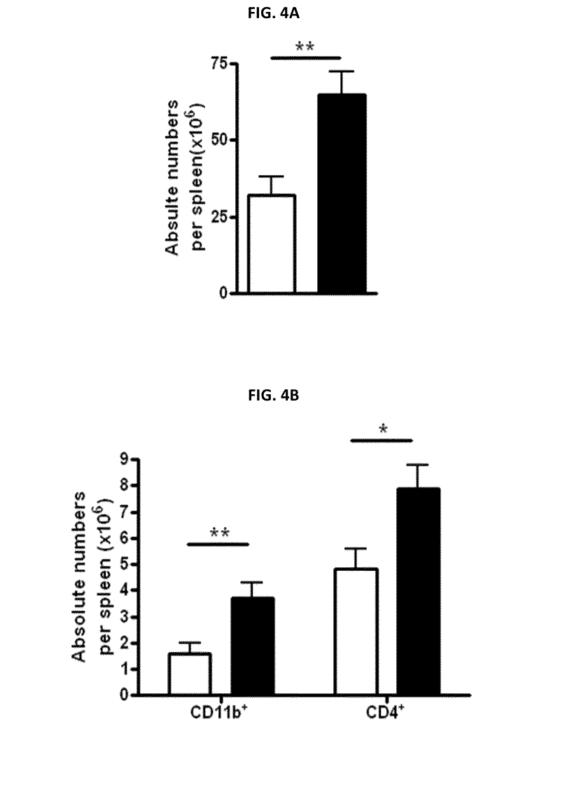

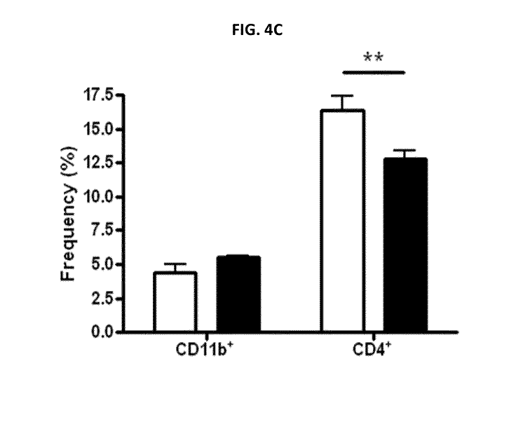

To evaluate the effects of DRα1-MOG-35-55 treatment on stroke-induced splenic atrophy, cell numbers in the spleen were counted in post-ischemic vehicle- and DRα1-MOG-35-55-treated mice. As expected, MCAO induced a significant decrease in spleen numbers in the Vehicle-treated group (FIG. 4A). Interestingly, viable cell counts were significantly increased in spleens of DRα1-MOG-35-55-treated versus Vehicle-treated mice (64.67×10⁶ vs. 32.11×10⁶ respectively, p<0.01) 96 hours after reperfusion. The increase in spleen cell numbers was reflected in the absolute numbers of CD11b⁺ cells (p<0.01) and CD4⁺ cells (p<0.05) (FIG. 4B). Although DRα1-MOG-35-55 treatment increased the cell numbers in the spleen, it reduced the frequency of CD4⁺ cells and did not change the frequency of the CD11b⁺ cells in the spleen (FIG. 4C).

### Example 6

### DRα1-MOG-35-55 Treatment Increases Frequency of Activated CD4+ Cells but does not Change Activation State of CD11b+ Cells in the Spleen after MCAO

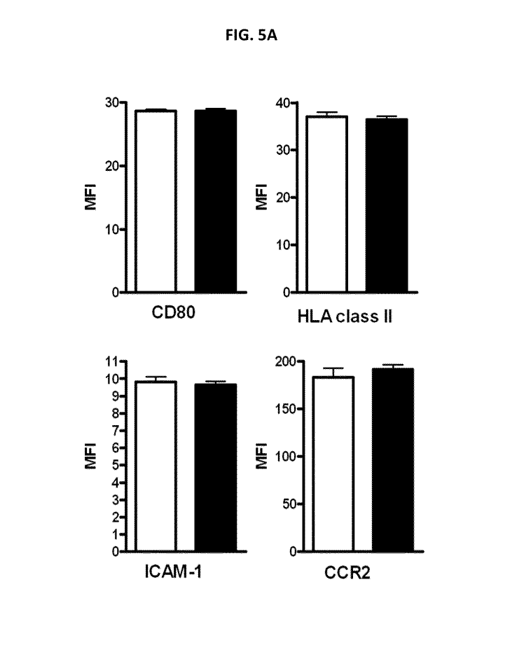

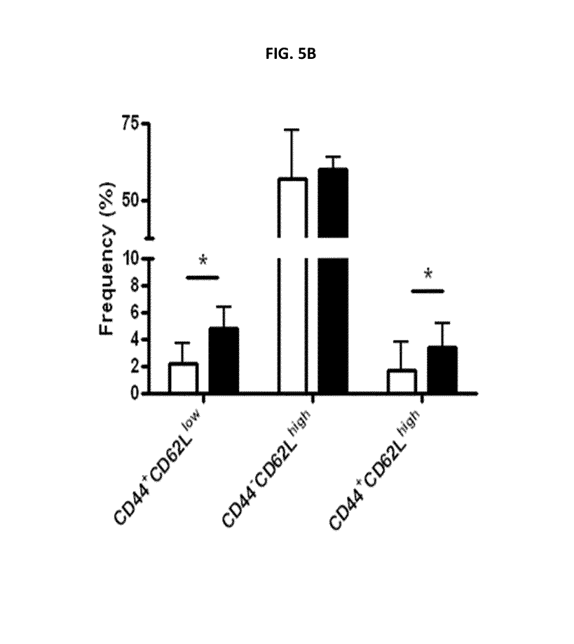

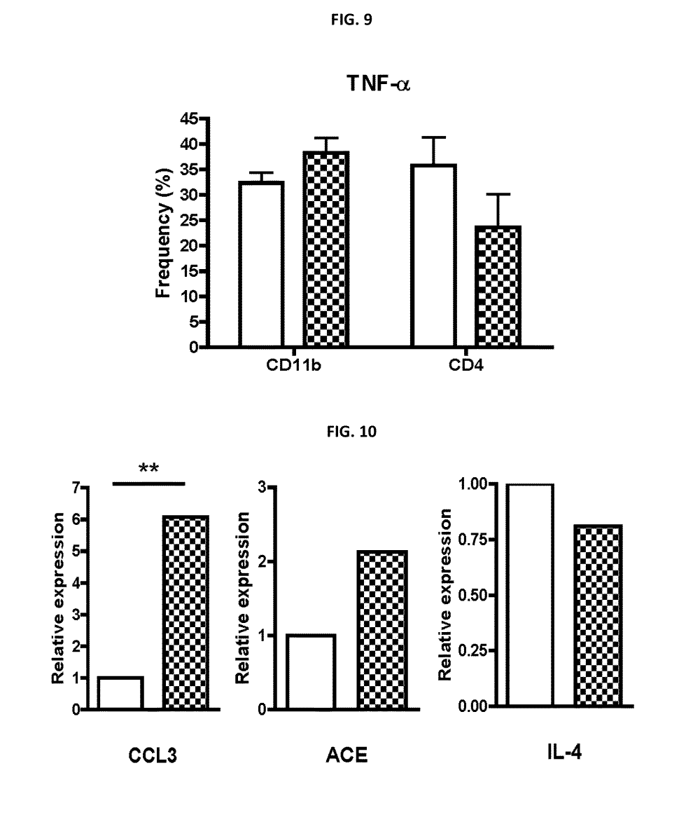

As shown in FIG. 5A, there were no differences in the level of expression of CD80, ICAM-1, HLA-DR, and CCR2 on CD11b⁺ cells between DRα1-MOG-35-55-versus vehicle-treated mice. Evaluation of CD4⁺ activation by the expression of CD44 and CD62L revealed that the frequency of both CD62L^{low} and CD62L^{high} activated CD4³⁰ cells in the spleen were increased after treatment with DRα1-MOG-35-55 compared with vehicle (p<0.05 for both; FIG. 5B). In addition, spleen cells from DRα1-MOG-35-55- or vehicle-treated mice were stimulated with PMA/Ionomycin for 4 hours and evaluated by flow cytometry for intracellular staining of TNF-α. There was no difference in production of TNF-α by CD4⁺ or CD11b⁺ cells in spleens from DRα1-MOG-35-55- or vehicle-treated mice (FIG. 9).

### Example 7

### DRα1-MOG-35-55 Treatment Affects the Immune Gene Expression Profile in the Spleen after MCAO

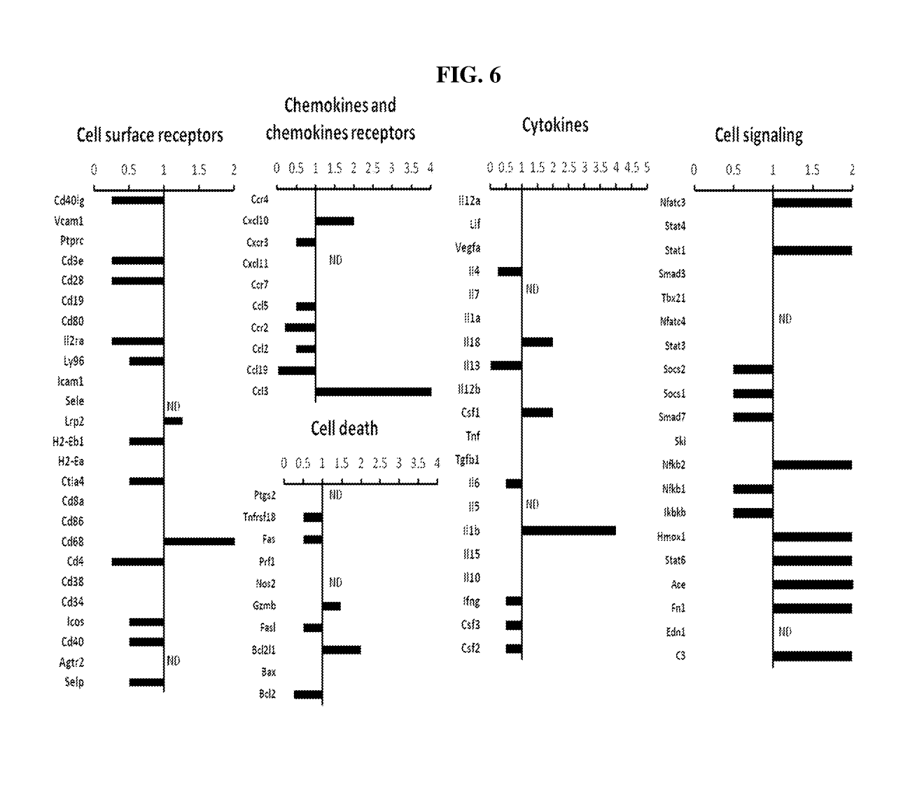

Messenger RNA was isolated from spleens of 6 vehicle-treated mice and 6 DRα1-MOG-35-55-treated mice. A real-time PCR assay was performed on pooled cDNA samples and expression levels from the DRα1-MOG-35-55-treated mice were analyzed relative to the Vehicle-treated mice (FIG. 6). The expression levels of 3 genes: IL-4, CCL3 and ACE were validated using individual samples (FIG. 10). Interestingly, the expression of several of the genes in the spleen, such as CCL3, IL-4, Stat6, ACE, FN1 and C3) had an opposite trend compared with their expression in the brain after DRα1-MOG-35-55 treatment. In addition, the expression of monocyte related genes, such as CD68, were increased in the spleen of DRα1-MOG-35-55-treated mice relative to Vehicle-treated mice. These expression results are in correlation to the absolute numbers of cells in the spleen.

### Example 8

### DRα1-MOG-35-55 Treats Stroke at a Lower Dosage

MCAO (60 min) was followed by treatment with 100 μl vehicle or 100 μg DRα1-MOG-35-55 given at 4, 24, 48 and 72 hour after MCAO. Brains were harvested at 96 hour and infarct volumes were measured as percentage of contralateral structure. *indicates p<0.05; **indicates p<0.01. Infarct volumes are shown in Table 1.

TABLE 1
Infarct volume with 100 μg/ml DRα1 MOG-35-55

| | CTX | CP | HMSPHR |
|---|---|---|---|
| Vehicle (n = 9) | 44.1 ± 2.9 | 65.1 ± 5.4 | 34.4 ± 3.7 |
| DRα1-MOG-35-55 | 25.8 ± 2.8 | 46.3 ± 5.3 | 18.6 ± 3.6 |
| (n = 11) | | | |

We claim:

1. A method of treating ischemic stroke in a subject, the method comprising:
administering to the subject a composition comprising an effective amount of DRα1-MOG-35-55 and a pharmaceutically acceptable carrier.

2. The method of claim 1, further comprising administering the composition to the subject after the onset of ischemia.

3. The method of claim 1, wherein the composition comprises a dose of 20-25 mg/kg of DRα1-MOG-35-55.

4. The method of claim 1, wherein the composition is formulated for subcutaneous or intravenous administration.

5. The method of claim 1, wherein the subject is human.

6. The method of claim 1, wherein the DRα1-MOG-35-55 comprises the amino acid sequence of SEQ ID NO: 1.

7. A composition for use in treating ischemic stroke in a subject comprising an effective amount of DRα1-MOG-35-55 and a pharmaceutically acceptable carrier.

8. The composition of claim 7, comprising a dose of 4-25 mg/kg of DRα1-MOG-35-55.

9. The composition of claim 7, formulated for subcutaneous or intravenous administration.

10. The composition of claim 7, wherein the DRα1-MOG-35-55 comprises the amino acid sequence of SEQ ID NO: 1.


SEQUENCE LISTING (1 sequence)

SEQ ID NO: 1 - protein, 120 aa, Artificial Sequence
  DRa1-MOG-35-55 protein
MEVGWYRSPFSRVVHLYRNGKGGGGSLVPRGSGGGGIKEEHVIIQAEFYLNPDQSGEFMFDFDGDEIFHVDMAKKETVWRLEEFGRFASFEAQGALANIAVDKANLEIMTKRSNYTPITN